(19) United States
(12) Patent Application Publication
(10) Pub. No.: US 2018/0134662 A1
(43) Pub. Date: May 17, 2018

(54) Aminopyridine Based Buffers with Wide Buffering Ranges Antibiotics and Myelin Disease Therapy

(71) Applicant: Thomas Daly, Arlington Heights, IL (US)

(72) Inventor: Thomas Daly, Arlington Heights, IL (US)

(21) Appl. No.: 15/871,554

(22) Filed: Jan. 15, 2018

Related U.S. Application Data
(63) Continuation-in-part of application No. 15/352,848, filed on Nov. 16, 2016, now Pat. No. 9,867,813.
(60) Provisional application No. 62/256,202, filed on Nov. 17, 2015.

(51) Int. Cl. C07D 213/81; A61P 25/00

(52) U.S. Cl. CPC C07D 213/81; C12Y 305/01098; A61P 25/00

(57) ABSTRACT

Amines and amine derivatives that improve the buffering range, and/or reduce the chelation and other negative interactions of the buffer and the system to be buffered. Aminopyridines channel blocking, combined with buffering and zwitterionic charge states make promising therapies for myelin diseases.

This is a continuation in part of application Ser. No. 15/352,848 filed Jul. 10, 2017 which claimed priority to provisional application No. 62/256,202 filed Nov. 17, 2015. Application Ser. No. 15/352,848 and 62/256,202 are hereby incorporated by reference in their entireties.

### BACKGROUND

### Field of the Invention

The present invention relates generally to the field of pyridine amines and more particularly to a classes of pyridine amines used as buffers in biological systems.

### Description of the Problem Solved by the Invention

Amines are very useful compounds in the buffering of biological systems. Each class of amine has various limitations which require choosing an amine based on multiple factors to select the best amine. For example, pH buffering range is typically most important, but issues of chelation, and pH range stability, and solubility also come into play. Additionally, buffers interact with the biological system beyond simple buffering. The pyridine amines function to complex with cations in addition to buffering. This property can be exploited to assist in pharmacological delivery systems, or as active pharmaceutical ingredients themselves.

### SUMMARY OF THE INVENTION

The present invention relates to amines and amine derivatives that improve the buffering range, and/or contribute to chelation. The reaction of amines or polyamines with various molecules to form polyamines with differing pKa's will extend the buffering range, derivatives that result in polyamines that have the same pKa yields a greater buffering capacity. Derivatives that result in zwitterionic buffers improve yield by allowing a greater range of stability.

### DESCRIPTION OF THE FIGURES

Attention is now directed to the following figures that describe embodiments of the present invention:

FIGS. 1A-1B teach the synthesis of zwitterionic buffers derived from 3,4-aminopyridine.

FIGS. 2A-2B show the derivation of zwitterionic buffers based on monochloroacetic acid and longer chain acrylates.

FIGS. 3A-3B teach the derivation of zwitterionic buffers from 2-aminopyridine, 3-aminopyridine, and 4-aminopyridine and aniline with 2-methyleneglutaric acid.

FIGS. 4A-4B teach synthesis of phosphonates and sulfonates from 3,4-aminopyridine.

FIGS. 5A-5B and FIG. 6A-6B show the synthesis of amides and carbamates from 3,4-aminopyridine.

FIGS. 7A-7B teach the synthesis of sulfonamides from 3,4-aminopyridine.

FIGS. 8A-8B teach the synthesis of dithiocarbamates from 3,4-aminopyridine.

FIGS. 9A-9B show the synthesis of alky amides of 3,4-aminopyridine.

FIGS. 10A-10B teach the synthesis of sulfonamides of 4-aminopyridine.

FIGS. 11-12 teach the synthesis of polyamides of 3,4-aminopyridine.

Several drawings and illustrations have been presented to aid in understanding the invention. The scope of the present invention is not limited to what is shown in the figures.

### DETAILED DESCRIPTION OF THE INVENTION

Combining amines with monochloroacetic acid (MCA) or sodium vinyl sulfonate (SVS) results in products are zwitterionic buffers that can buffer in both acidic and basic pH conditions. A limited number amines are currently used for this purpose, such as, tromethamine and ammonia. The reaction of amines, alcohols, and aminoalcohols with acrylonitrile (via the Michaels Addition), followed by reduction results in amines and polyamines that have a broad buffering range. The further derivatization of the amines and polyamines with MCA and SVS yields a further crop of amine buffers with desirable properties. One skilled in the art will recognize that MCA and sodium monochloroacetic acid (SMCA) can be used interchangeably. Furthermore, the buffers taught herein, and their pharmacologically acceptable salts, act as potassium channel blockers and many will behave as MAOI, monoamine oxidase inhibitors, making them potential therapeutic agents for demyelinating diseases, Alzheimer's disease, Parkinson's disease, multiple sclerosis, and other neurological disorders such as ALS. Furthermore, several molecules taught here as part of this invention are expected to promote the formation of astrocytes. Astrocytes are a key component of maintaining function and healing of spinal cord injuries, particularly those that result in a less than complete severing of the spinal cord, but that result in a removal of myelin from part of the neuron. In this case, the potassium ions that propagate the action potential are able to leak out of the neuron. The use of a potassium channel blocker, such as those taught here, along with an astrocyte promoter greatly improves neural function.

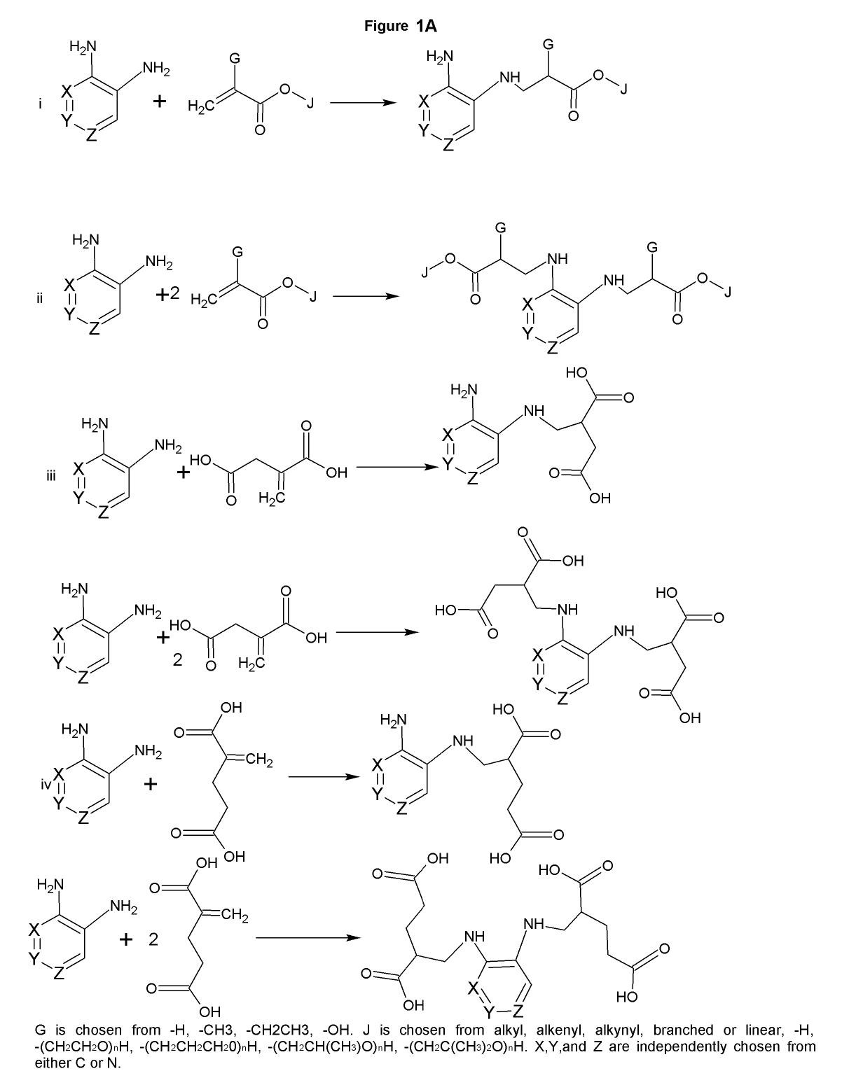

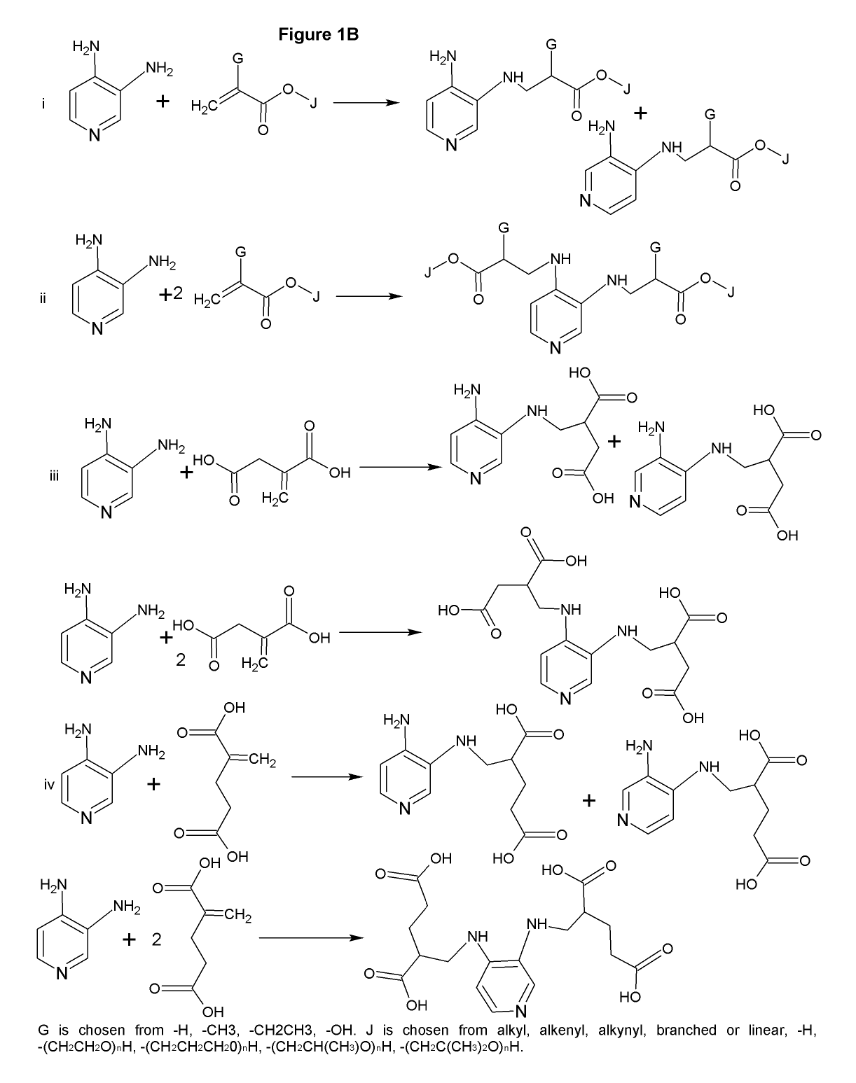

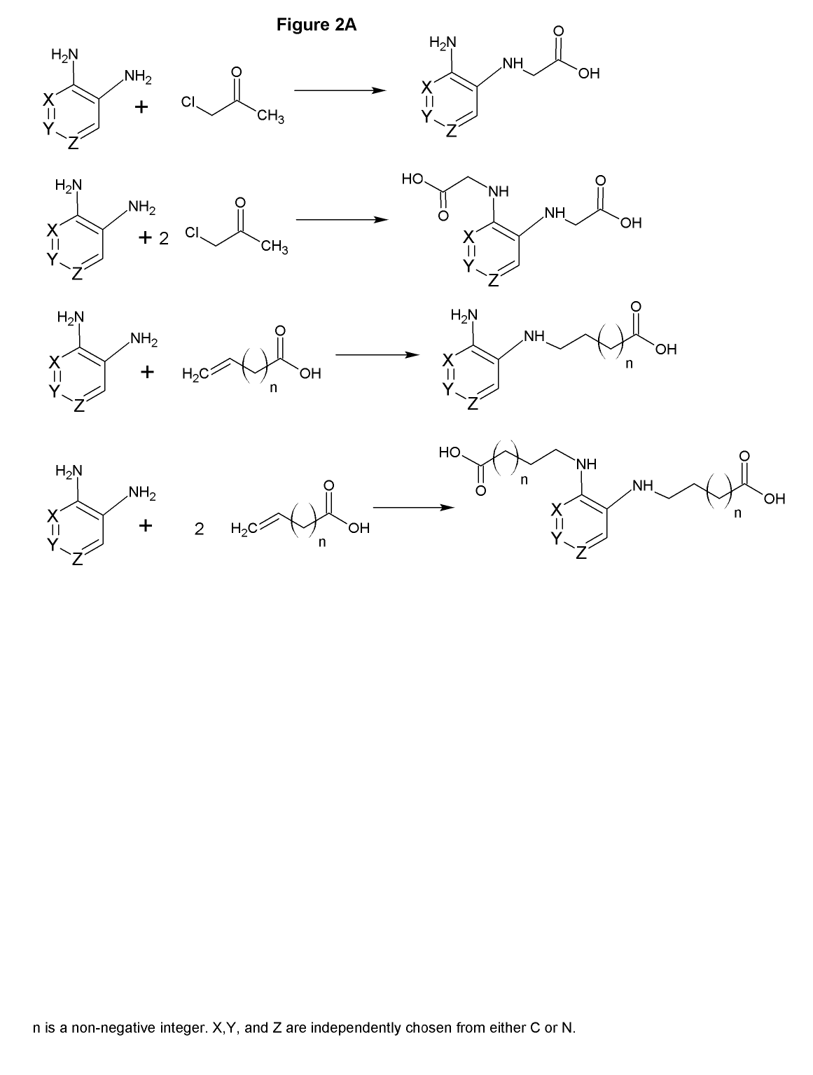

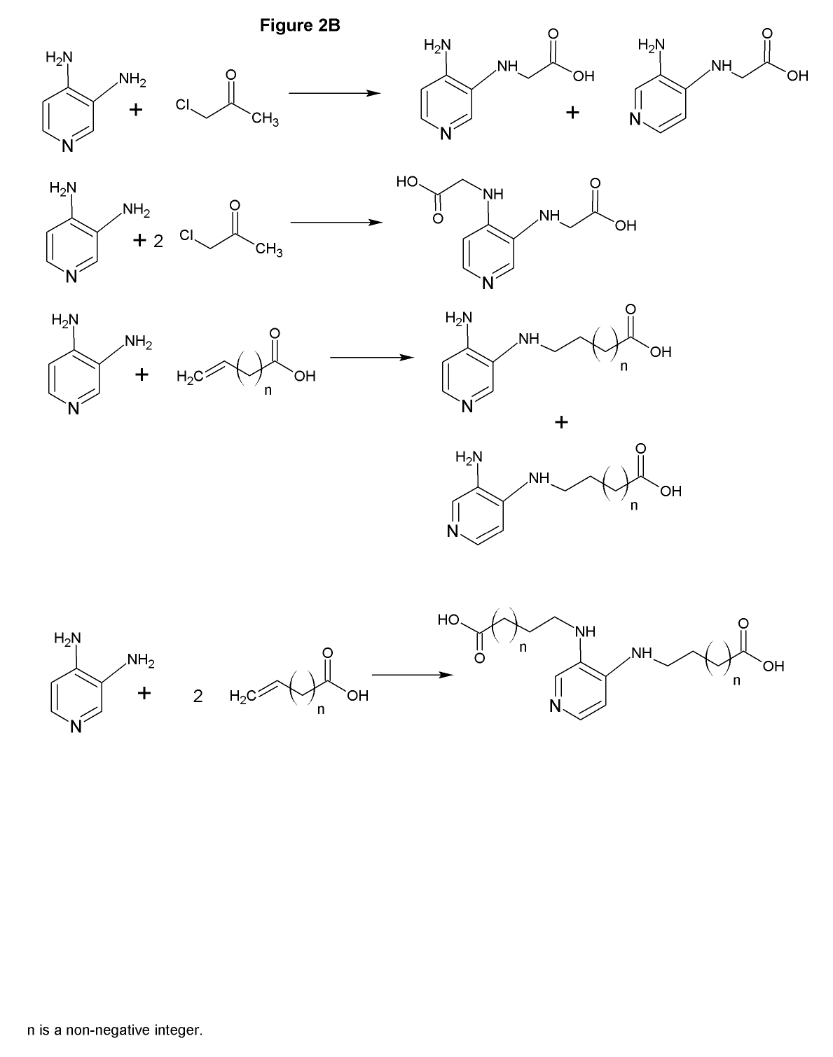

FIGS. 1 and 2 teach the synthesis of zwitterionic buffers of 3,4-diaminopyridine. Line i teaches the synthesis of a mono substitution on only one primary amino group. Line ii teaches the synthesis where each primary amino group is monosubstituted to the secondary amine. By utilizing HCl and methanol as the reaction media, the tertiary amines can be formed. The tertiary amines are simply the form where each primary amine group is disubstituted. The Michael addition products need not be symmetric, where the Michael adduct is all the same. The additions can be sequentially to make asymmetric products. The order of addition is such that one primary amino group reacts with the Michael adduct, then the second primary amine. Adding HCl and methanol, then adding a third equivalent of a Michael adduct will form a tertiary amine to one of the original primary amines, and finally the last amino hydrogen will react to from the only tertiary amine containing product. While not generally explicitly shown in subsequent figures, this principle holds throughout the invention disclosure.

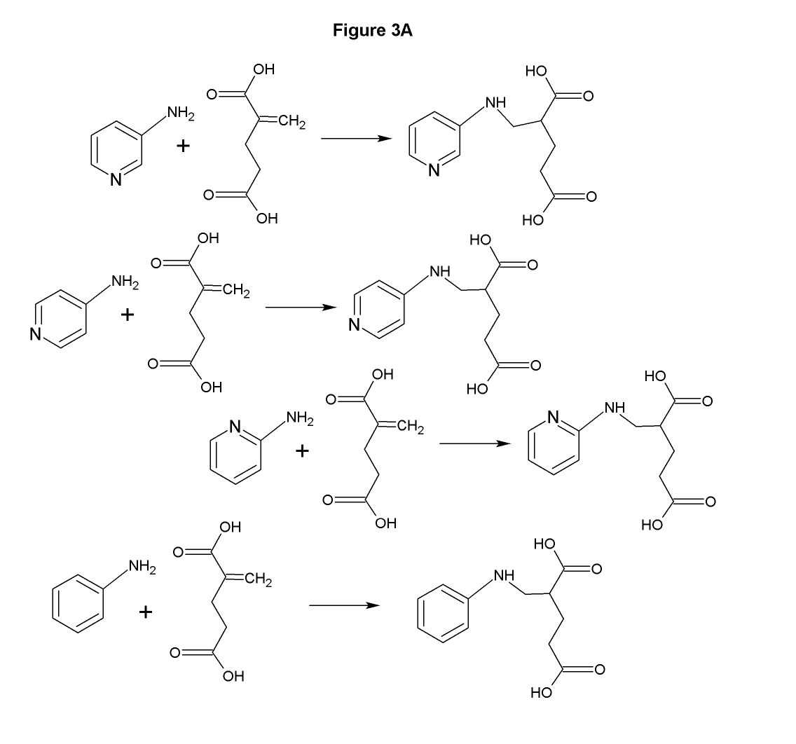

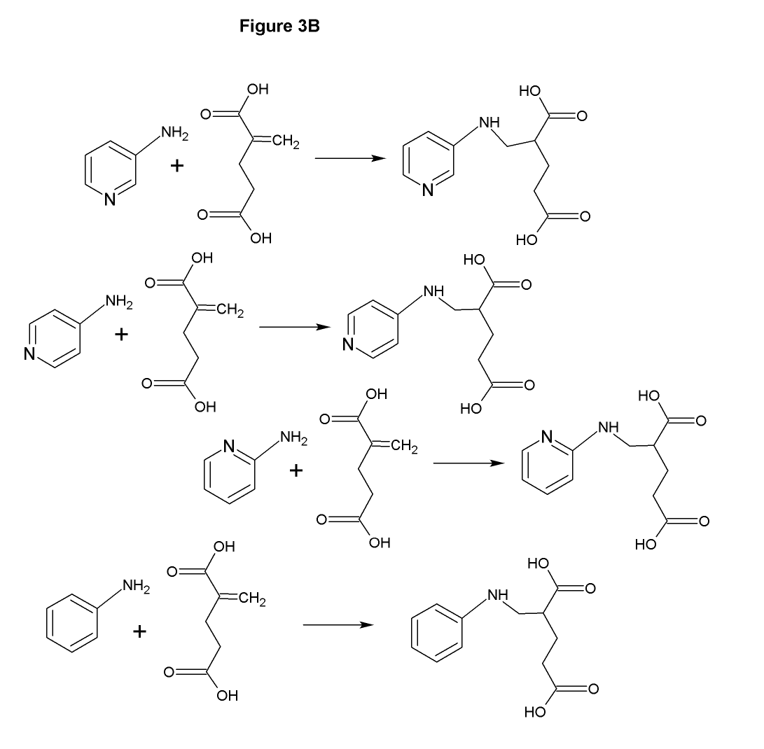

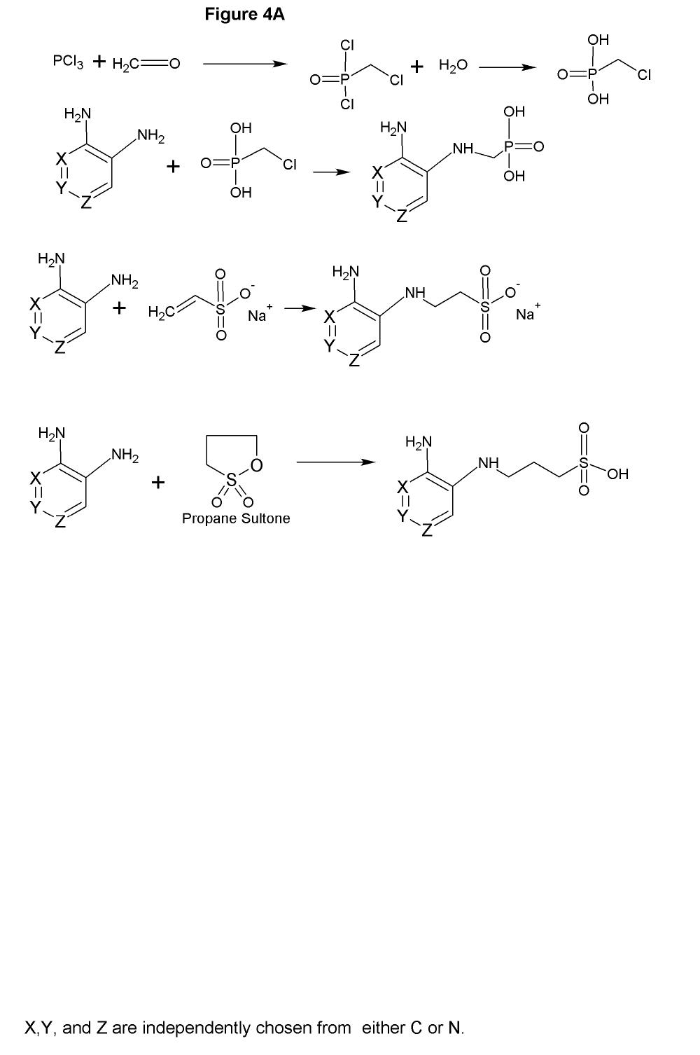

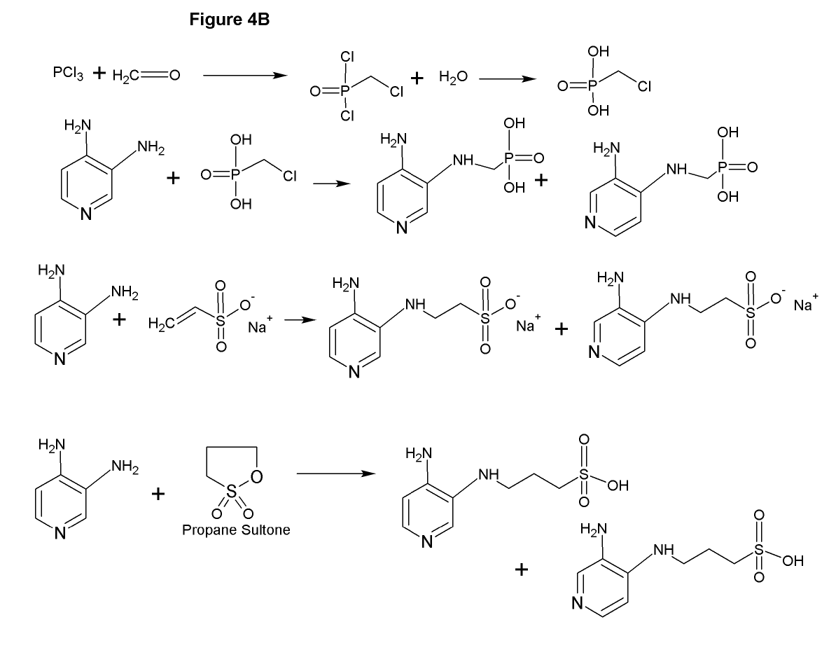

FIG. 3 teaches the zwitterionic buffers of aminopyridines and 2-methyleneglutaric acid. These products act as potassium channel blockers, but are also expected to promote the generation of astrocytes, and thus greater neurological function. FIG. 4 teaches the synthesis of phosphonate and sulfonate buffers. As before, the figures only show the 1:1 molar reaction, but both reactive sites on the two primary amines may be reacted, yielding up to four sites. The simplest beyond the single mono substituted products are the disubstituted products where each amine group undergoes one addition to form the secondary amine. For the propane sultone product, the higher sultone analogs, such as butyl sultone are part of this invention as well.

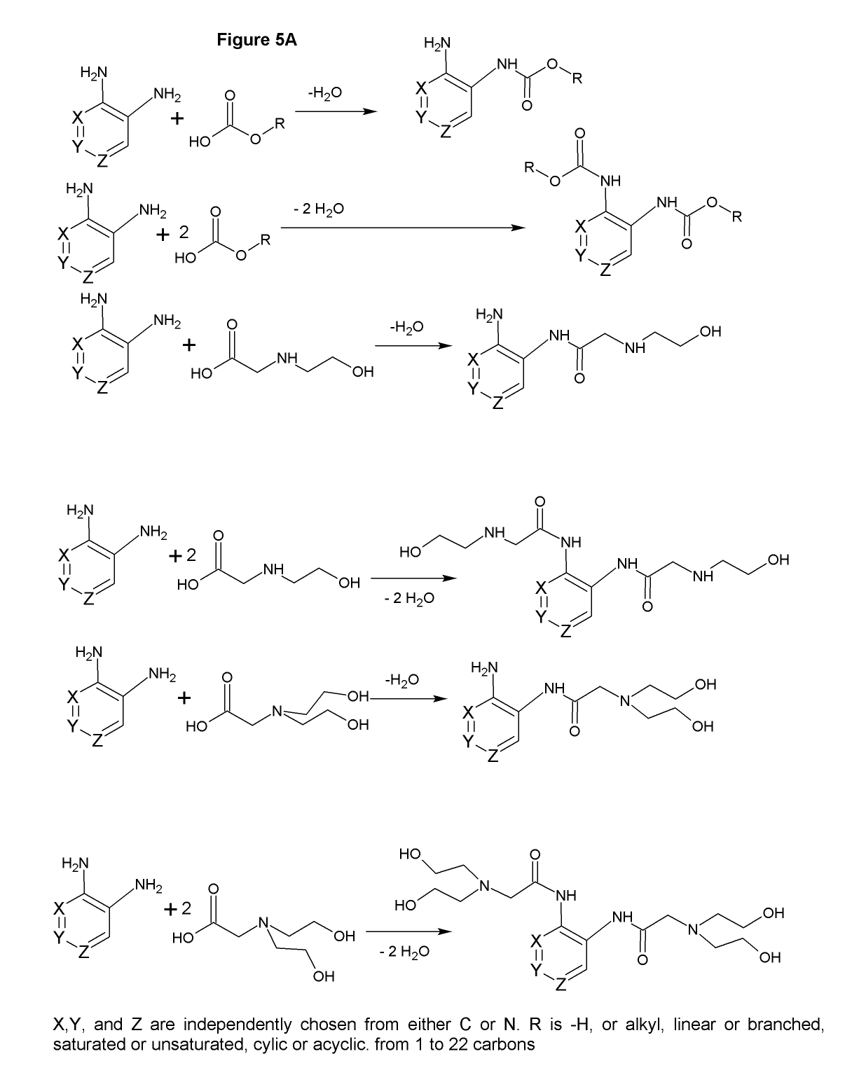

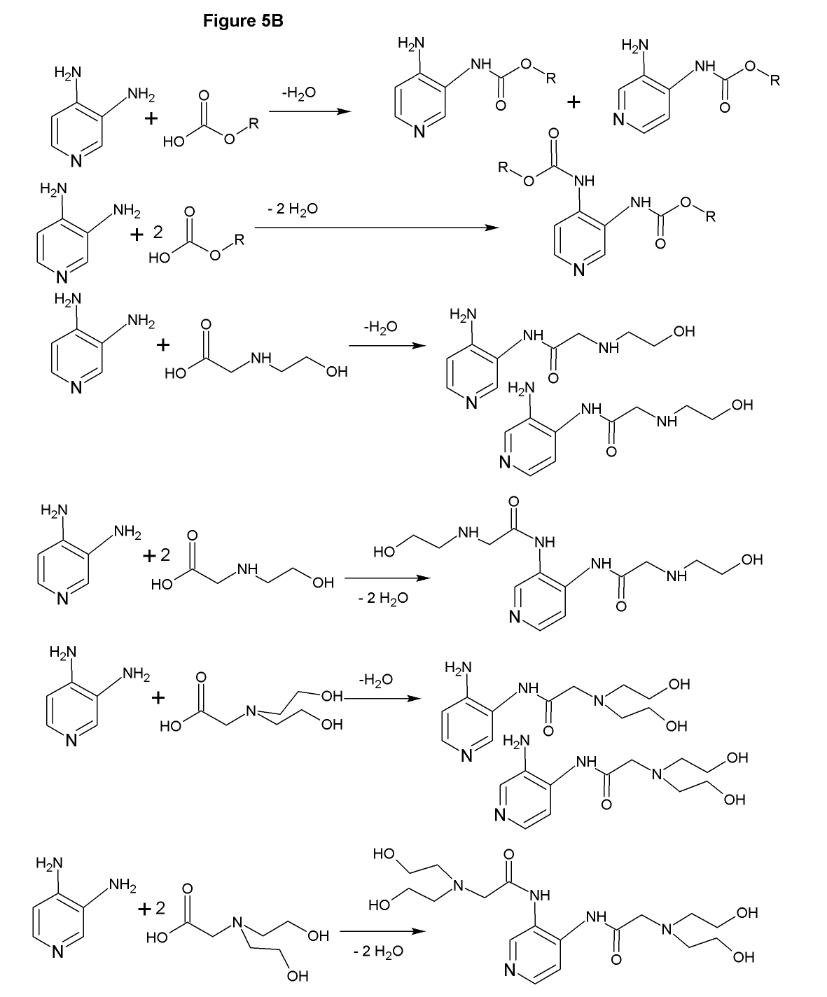

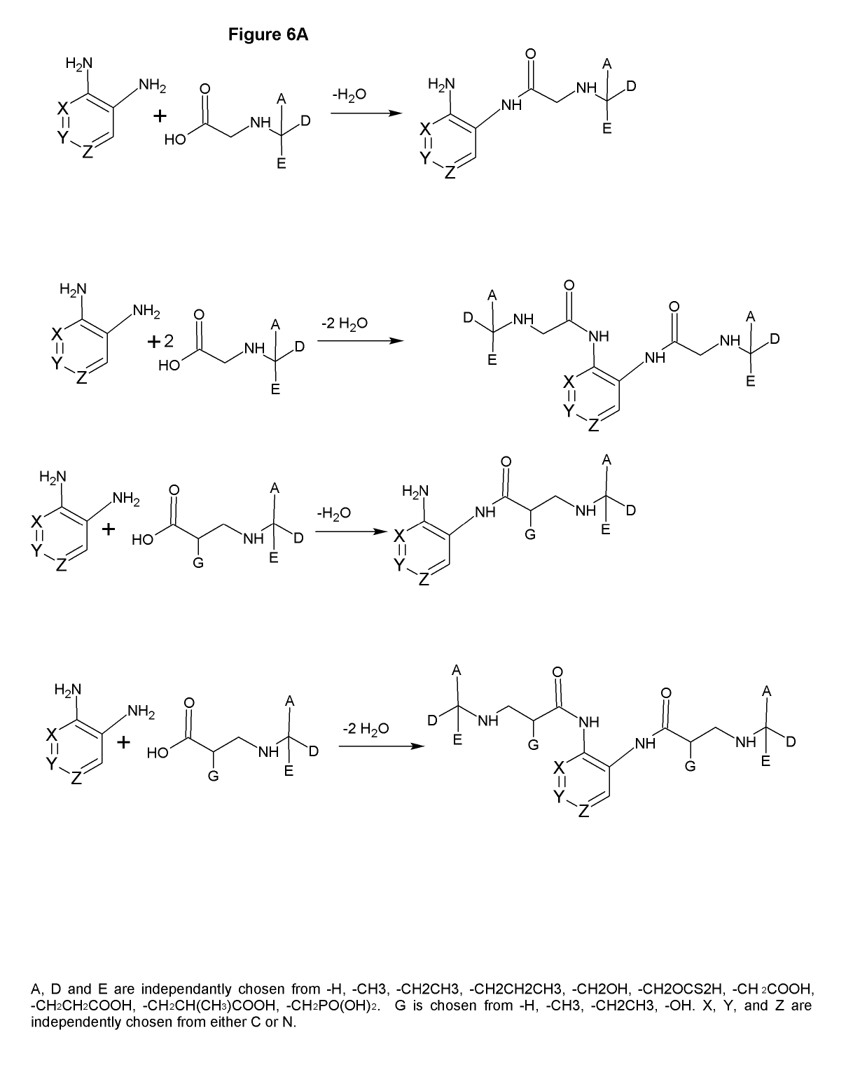

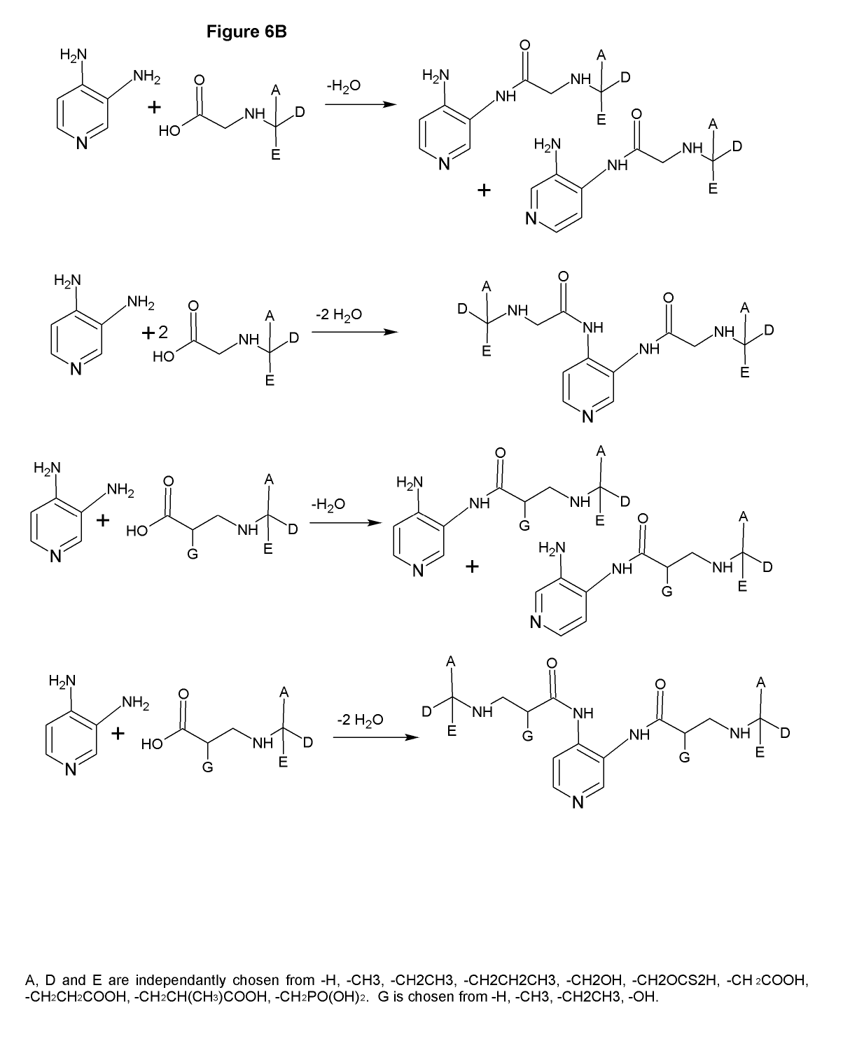

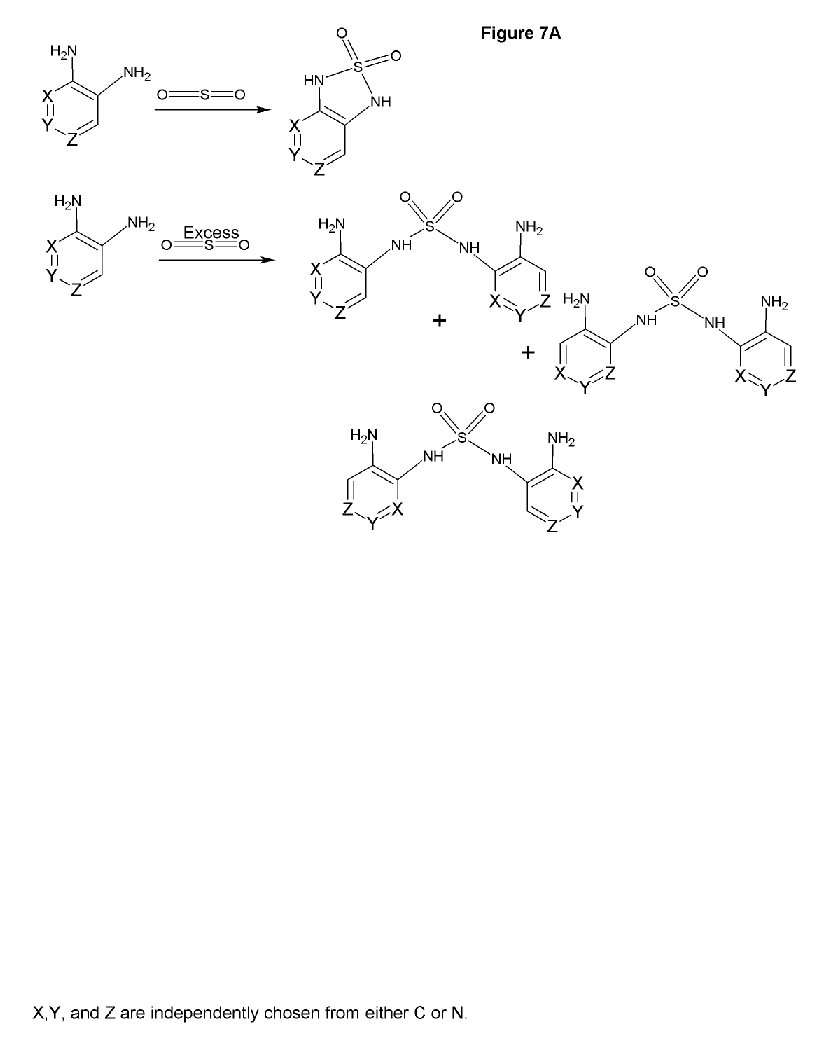

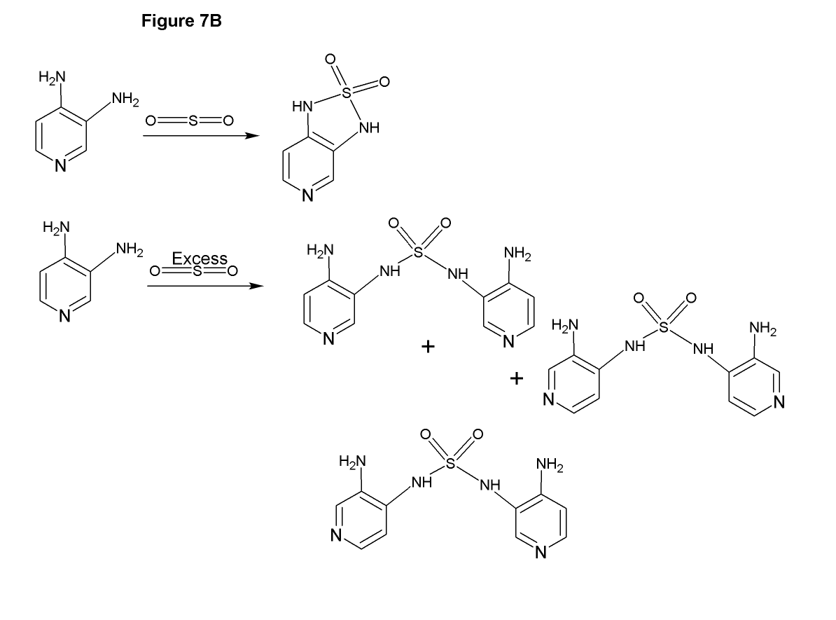

FIG. 5 teaches the synthesis of amides and carbamates that are useful as potassium channel blockers. Monethanol amine and diethanolamine analogues are taught and shown as symmetric additions. This need not be the case. Asymmetric additions are part of this invention. FIG. 6 expands on FIG. 5 to include a wider range of amides and carbamates. Again, while the figure shows a symmetric addition, asymmetric additions, where the constituent groups, A, D, E are not the same for the different nitrogens, are within the scope of this invention. FIG. 7 teaches the synthesis of sulfamides based in 3,4-aminopyridine.

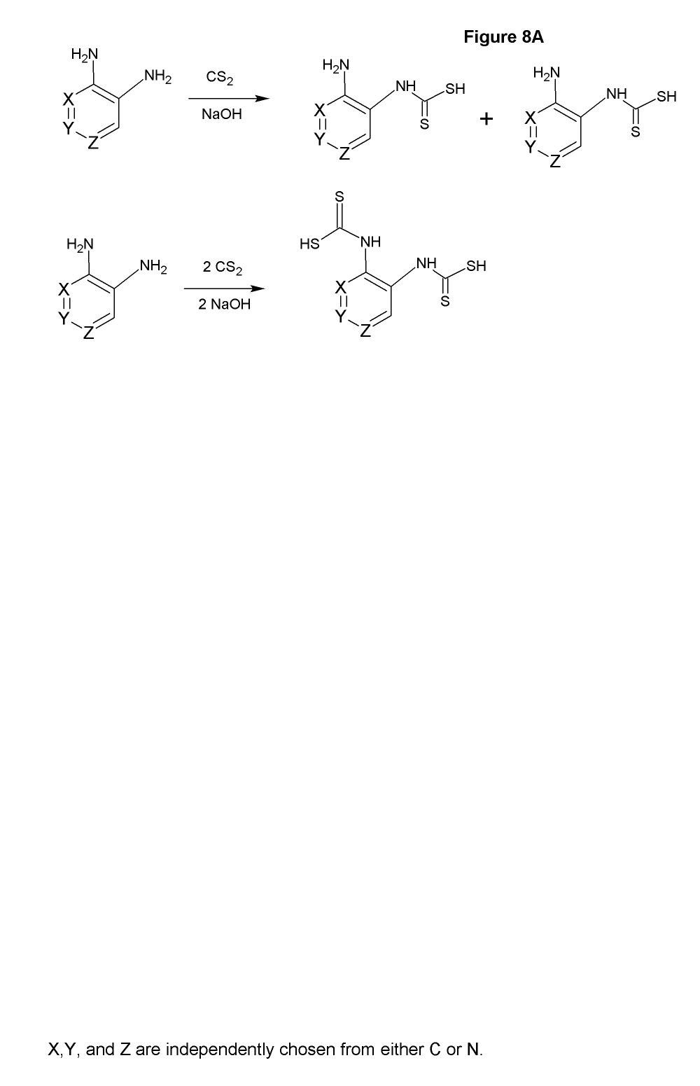

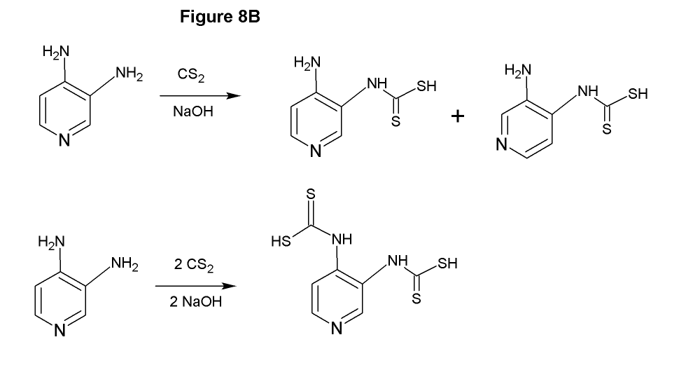

FIG. 8 teaches the synthesis of dithiocarbamates based on 3,4-aminopyridine. While caustic is shown as the basicity agent, any other basicity agent may be used, including, but not limited to other mineral bases, such as KOH, and tertiary amines. methyldicoco amine is particularly useful as the resulting product exhibits much greater antimicrobial properties than the sodium or potassium derived forms. This is particularly interesting in agriculture and the methyldicoco amine acts as an adjuvant as well. The figure shows the dithiocarbamates as free molecules, however, the free molecules are not stable and must be made and stored as the salts. The representation as free molecules and not salts is to simplify the structure and to not limit the invention to any particular salt. One skilled in the art will recognize all salts as part of the invention.

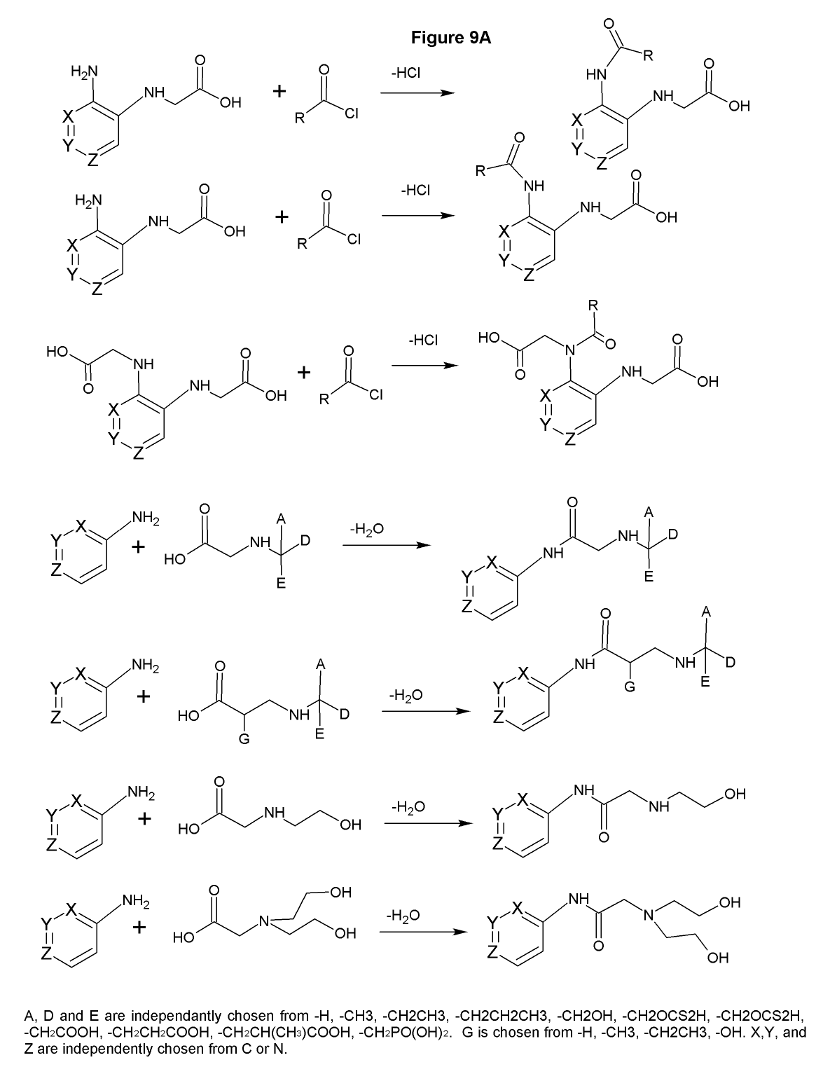

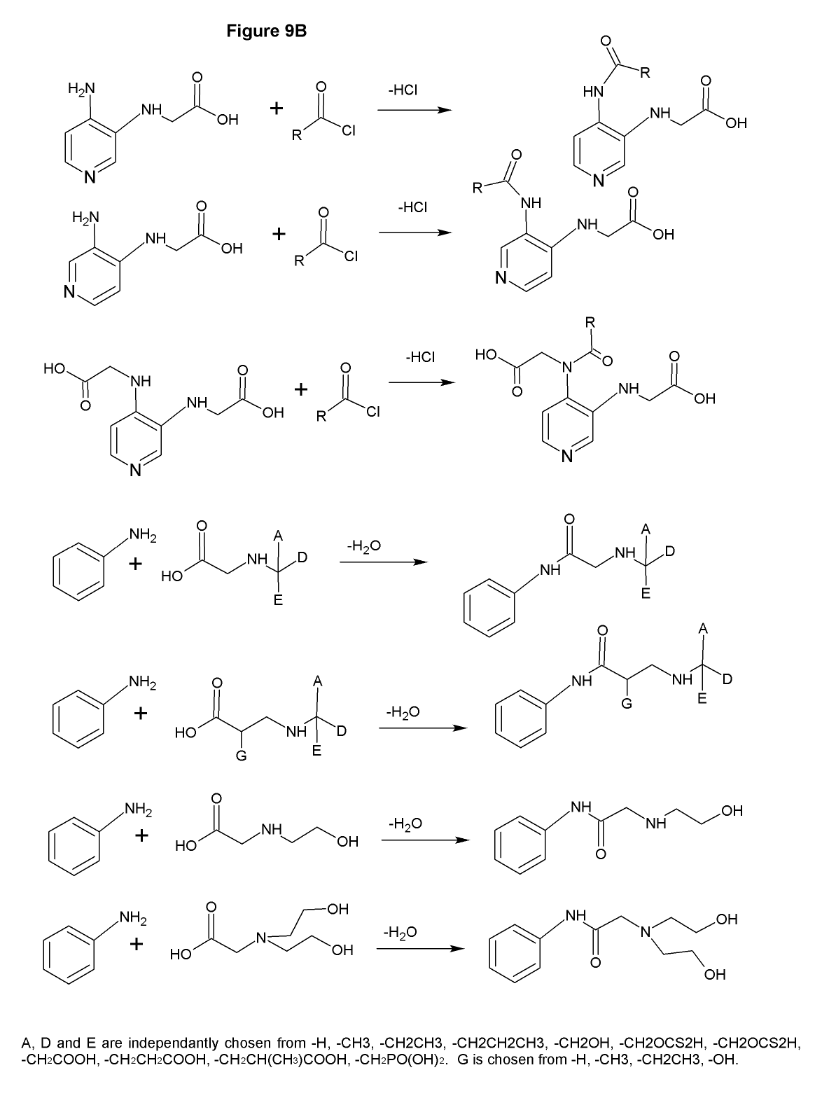

FIG. 9 teaches the addition of an acid chloride to form an amide group to the zwitterionic buffers. The addition of the acid chloride to form the amide allows for the adjustment of water solubility and blood brain barrier penetration. R is any alkyl chain, linear or branched, saturated or unsaturated, cyclic or acyclic. A second, symmetric or asymmetric addition of acid chloride can be made to further enhance the shift in solubility. Also taught are less water soluble amides from aniline.

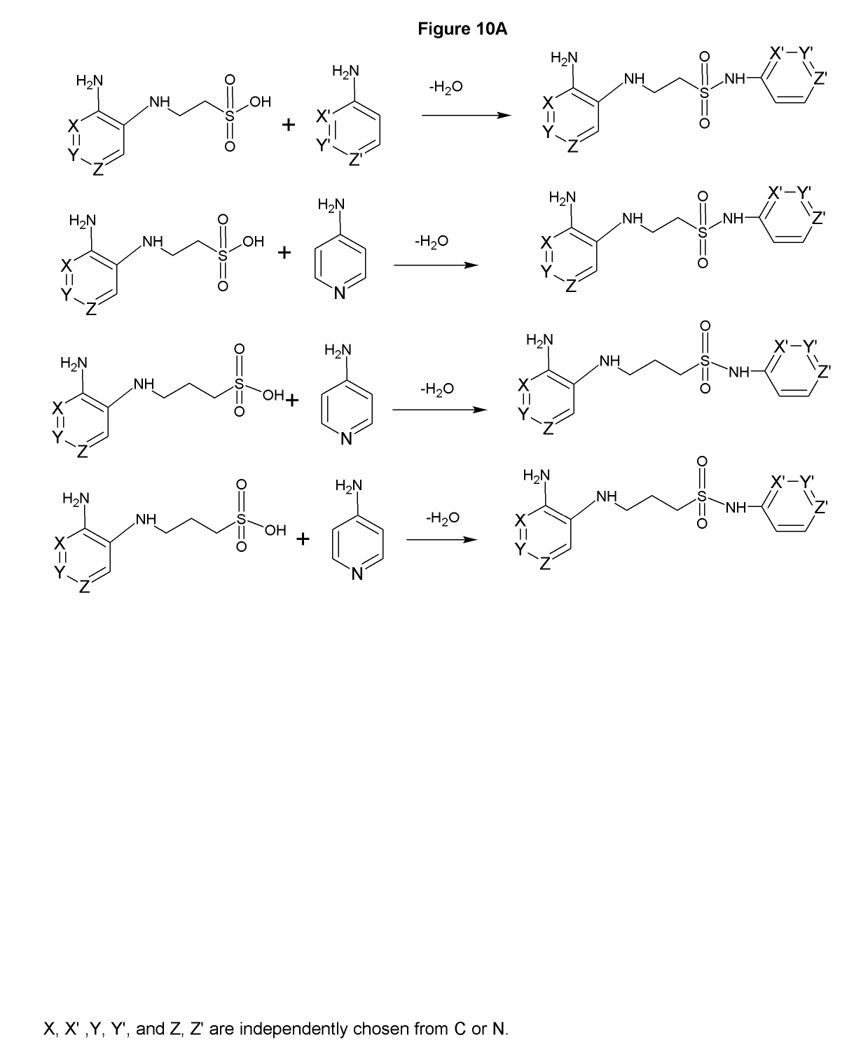

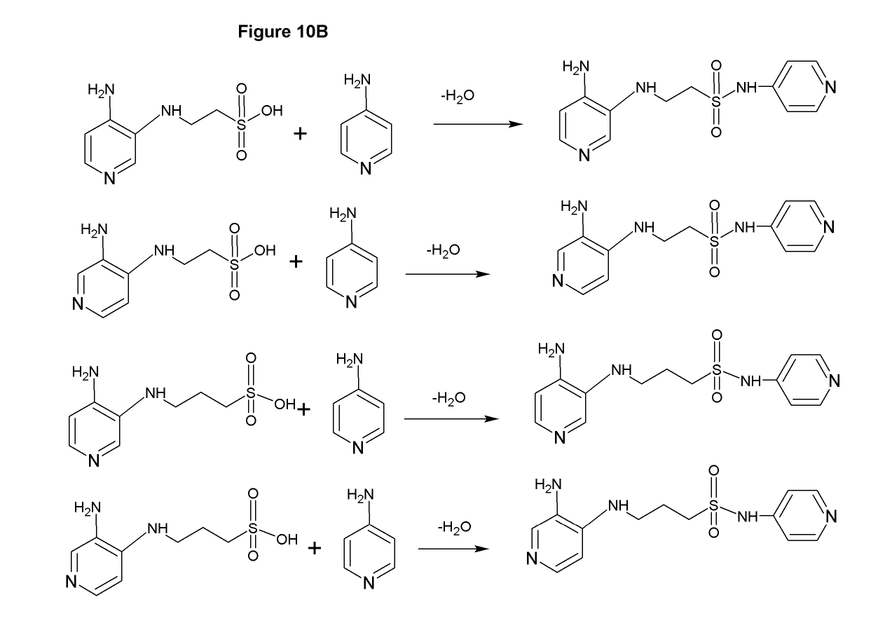

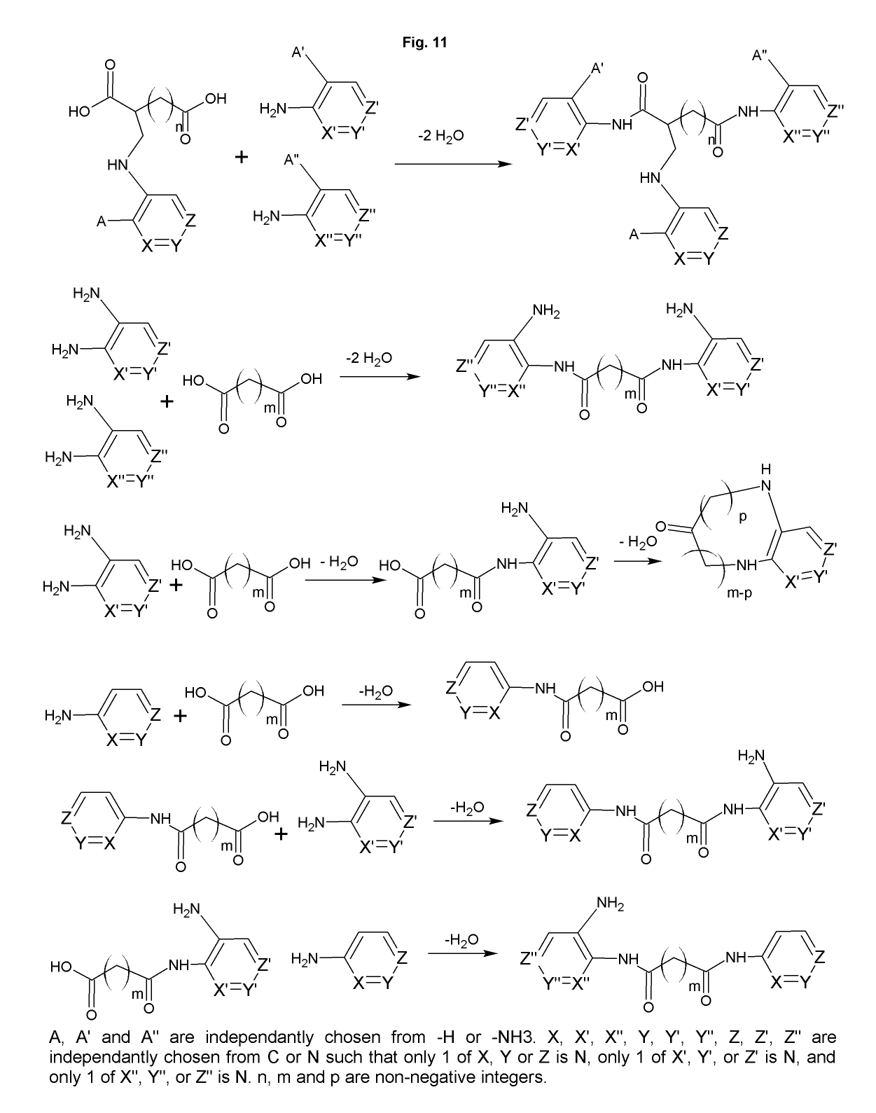

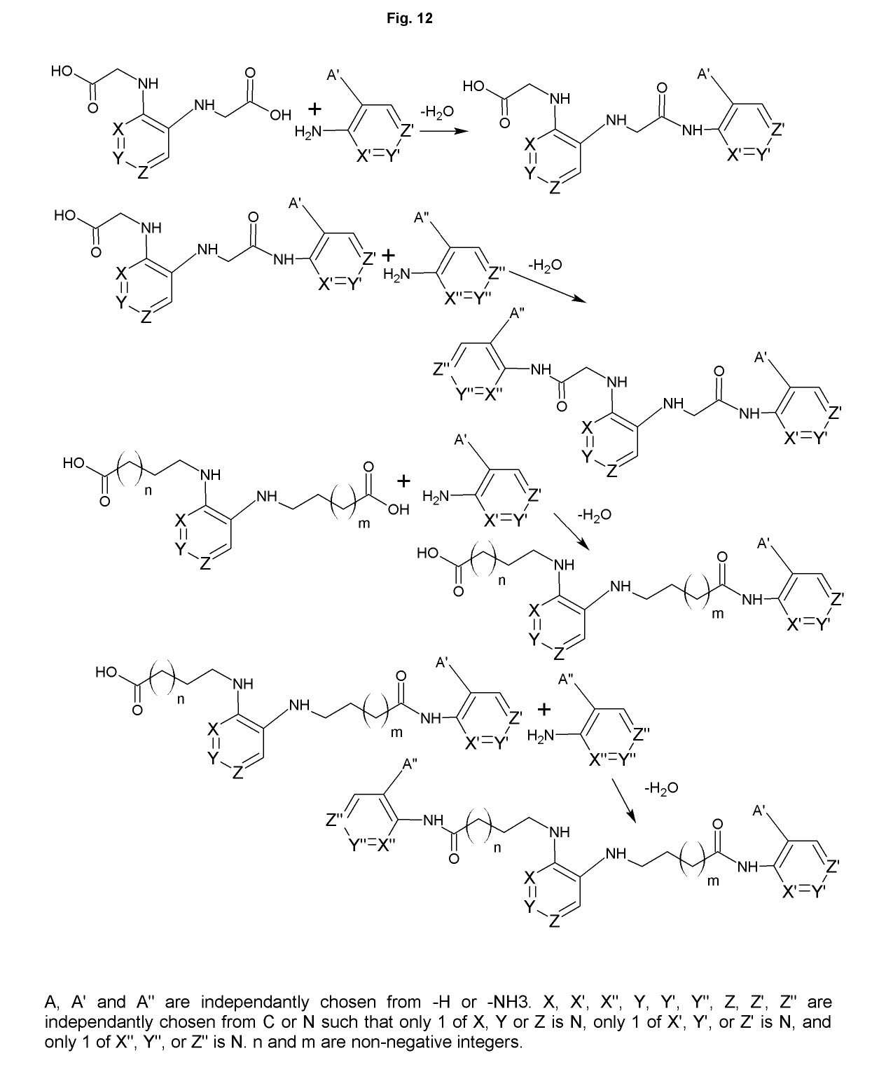

FIG. 10 teaches sulfonamides from the sulfonates of FIG. 7 and 4-aminopyridine. The sulfonamides of the higher sulfonates from FIG. 7 are also part of the invention as are the sulfonamides of 2-aminopyridine and 3-aminopyridine in place of 4-aminopyridine. The analogous products where both primary amine groups of 3,4-diaminopyridine are reacted with either sodium vinyl sulfonate or a sultone to make the disulfonate of 3,4-diaminopyridine, can then be made into the corresponding disulfonamide with either 2-aminopyridine, 3-aminopyridine, 4-aminopyridine, 3,4-diaminopyridine or any combination thereof. FIGS. 11-12 teach a series of polyamides based on 3,4-diaminopyridine as well as aminopyridines in general. The polyamides are expected to be potential treatments for cancer. The believed, but unconfirmed mode of action is thought to be histone deacetylase inhibition.

Several descriptions and illustrations have been presented to enhance understanding of the present invention. One skilled in the art will know that numerous changes and variations are possible without departing from the spirit of the invention. Each of these changes and variations are within the scope of the present invention.

I claim:

1) A histone deacetylase inhibitor of the following structure: 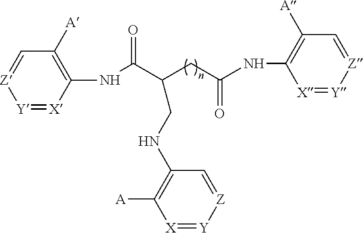
where A, A′ and A″ are independently chosen from —H or —NH3, X, X′, X″, Y, Y′, Y″, Z, Z′, Z″ are independently chosen from C or N such that only 1 of X, Y or Z is N; only 1 of X′, Y′, or Z′ is N; and only 1 of X″, Y″, or Z″ is N, where n is a non-negative integer.

2) A histone deacetylase inhibitor of the following structure: 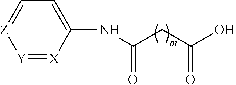
where, X, Y, and Z are selected from C or N; where only one of X, Y, or Z may be N, where m is a non-negative integer.

3) A histone deacetylase inhibitor of the following structure: 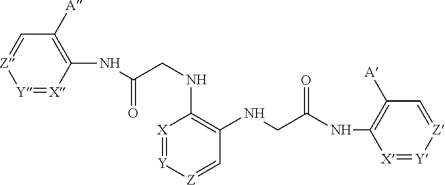
where A′ and A″ are independently chosen from —H or —NH3; X, X′, X″, Y, Y′, Y″, Z, Z′, Z″ are independently chosen from C or N such that only 1 of X, Y or Z is N; only 1 of X′, Y′, or Z′ is N; and only 1 of X″, Y″, or Z″ is N.
